(19) United States
(12) Patent Application Publication
(10) Pub. No.: US 2023/0363729 A1
(43) Pub. Date: Nov. 16, 2023

(54) STENT DELIVERY SYSTEM AND STENT DELIVERY METHOD

(71) Applicant: OLYMPUS CORPORATION, Tokyo (JP)

(72) Inventors: Kenji MURAKAMI, Hino-shi (JP); Kazuhiro GONO, Sagamihara-shi (JP); Genri INAGAKI, Fuchu-shi (JP); Yuji SAKAMOTO, Kunitachi-shi (JP); Nao INOUE, Isehara-shi (JP); Shohei HEMMI, Tokyo (JP); Hiroyuki MINO, Westborough, MA (US); Hirokazu HORIO, Allentown, PA (US); Atanaska GOSPODINOVA, Hamburg (DE); Shintaro INOUE, Westborough, MA (US)

(73) Assignee: OLYMPUS CORPORATION, Tokyo (JP)

(21) Appl. No.: 18/196,508

(22) Filed: May 12, 2023

Related U.S. Application Data
(60) Provisional application No. 63/341,526, filed on May 13, 2022.
(60) Provisional application No. 63/341,534, filed on May 13, 2022.

(51) Int. Cl. A61B 6/12; A61B 6/00; A61F 2/966; G16H 40/63

(52) U.S. Cl. CPC A61B 6/12; A61B 6/461; A61F 2/966; G16H 40/63; A61B 1/018

(57) ABSTRACT

A stent delivery system includes: a stent delivery device configured to carry a stent to a stenosis and indwell the stent; an observation device configured to observe the stenosis; and one or more processors, each comprising hardware, the one or more processors being configured to: acquire an observation image from the observation device; and determine a target placement position for placing the stent in the stenosis based on the observation image.

### CROSS-REFERENCE TO RELATED APPLICATIONS

The present application claims priority based on U.S. Patent Provisional Application No. 63/341,534 provisionally filed in the United States on May 13, 2022, and U.S. Patent Provisional Application No. 63/341,526 provisionally filed in the United States on May 13, 2022, the contents of each of which are incorporated herein by reference.

### BACKGROUND

The present disclosure relates to stent delivery systems and stent delivery methods.

A stent delivery device is used when a self-expanding stent is placed in a stenosis or occlusion (hereinafter referred to as “stenosis, etc.”) that occurs in a lumen. In a conventional stent delivery device, a stent is housed in a gap between an inner sheath and an outer sheath, and the stent is exposed and expanded by retracting the outer sheath with respect to the inner sheath. The stent is left in the lumen by removing the inner sheath from the stent.

### SUMMARY

A stent delivery system and a stent delivery method that facilitate placement of a stent at the desired location are desired.

Based on the above circumstances, the present disclosure aims to provide a stent delivery system and a stent delivery method that facilitate placement of a stent at a desired position.

In order to solve the above problems, this disclosure proposes the following means.

A stent delivery system according to a first aspect of the present disclosure includes: one or more processors, each comprising hardware, the one or more processors being configured to: acquire an observation image from the observation device; and determine a target placement position for placing the stent in the stenosis based on the observation image.

A stent delivery method according to a second aspect of the present disclosure is a method for placing a stent with a stent delivery device, the method comprising: acquiring an observation image from an observation device that observes the stenosis; determining a target placement position for placing the stent in a stenosis based on the observation image.

A control device according to a third aspect of the present disclosure is a control device for placing a stent with a stent delivery device, the control device comprising: one or more processors comprising hardware, the one or more processors being configured to: determine a target placement position for placing the stent in a stenosis based on an observation image acquired from an observation device that observes the stenosis.

### BRIEF DESCRIPTION OF THE DRAWINGS

FIG. 1 is a diagram showing the overall configuration of an endoscope system according to a first embodiment.

FIG. 2 is a diagram showing a stent delivery device of the endoscope system.

FIG. 3 is a functional block diagram of a drive device of the endoscope system.

FIG. 4 is a diagram showing a delivery device driving device of the drive device.

FIG. 5 is a functional block diagram of a video control device of the endoscope system.

FIG. 6 is a diagram showing a bile duct in which a stent is placed.

FIG. 7 is a follow-up chart showing a step for placing the stent.

FIG. 8 is a control flowchart of the control device of the endoscope system in the stent placement step.

FIG. 9 is an X-ray image acquired by an observation image acquisition step.

FIG. 10 shows the stent delivery device moved to the vicinity of the stenosis.

FIG. 11 is a display image of the display device of the endoscope system on which navigation information is displayed.

FIG. 12 is a display image of the display device in which other navigation information is displayed.

FIG. 13 is a display image of the display device in which other navigation information is displayed.

FIG. 14 is a diagram for explaining an automatic placement step.

FIG. 15 is a diagram showing the stent being placed in the automatic placement step.

FIG. 16 is a diagram showing the stent placed in the automatic placement step.

### DETAILED DESCRIPTION

### First Embodiment

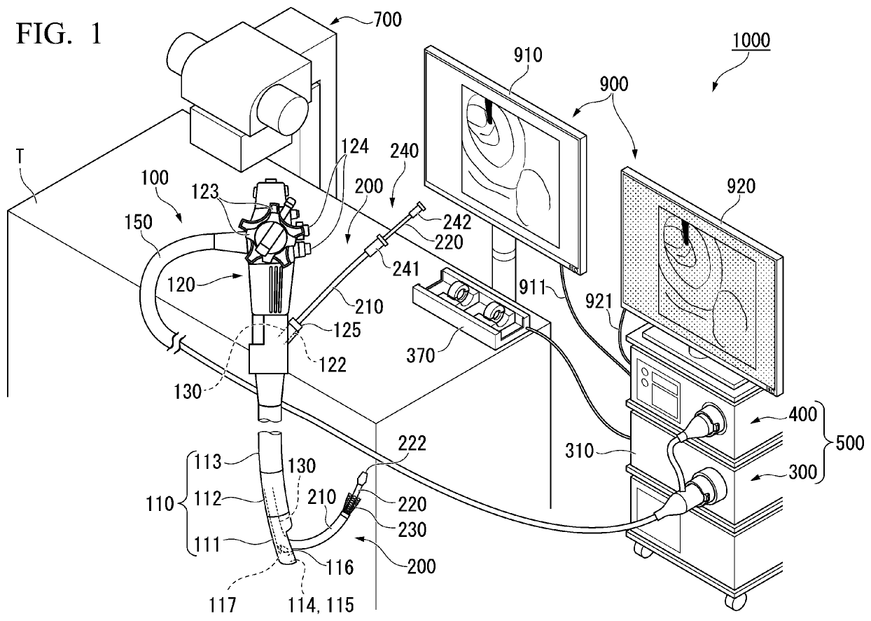

An endoscope system 1000 according to the first embodiment of the present disclosure will be described with reference to FIGS. 1 to 16. FIG. 1 is a diagram showing the overall configuration of an endoscope system 1000.

### [Endoscope System 1000]

An endoscope system (stent delivery system) 1000 is a medical system for observing and treating the inside of a patient's body. The endoscope system 1000 includes an endoscope 100, a stent delivery device 200, a drive device 300, a video control device 400, an observation device 700, and a display device 900.

The endoscope 100 is a device that is inserted into a patient's lumen to observe and treat an affected area. The endoscope 100 is connected to the drive device 300 and the video control device 400 via a universal cord 150.

The stent delivery device 200 is a device that is inserted into the treatment instrument channel 130 of the endoscope 100 to leave the stent 230 in the patient's lumen, as shown in FIG. 1.

The drive device 300 is connected to the endoscope 100 via the universal cord 150. The drive device 300 drives a built-in pump or the like based on an operation input to the operation portion 120 of the endoscope 100 to cause the endoscope 100 to perform air supply and suction.

The drive device 300 has a delivery device driving device 370 to which the operation portion 240 of the stent delivery device 200 is detachably connected.

The video control device 400 is detachably connected to the video control cable 670. The video control device 400 is connected to the endoscope 100 via the universal cord 150 and acquires captured images from the endoscope 100. The video control device 400 causes the display device 900 to display captured images acquired from the endoscope 100 and GUI images and CG images for the purpose of providing information to the operator.

The drive device 300 and the video control device 400 constitute a control device 500 that controls the endoscope system 1000. The control device 500 may further includes peripherals such as a video printer. The drive device 300 and the video control device 400 may be an integrated device.

The observation device 700 is a known X-ray fluoroscopy device that emits X-rays from outside the body to observe a patient. The observation device 700 may include a device for observing a patient by CT (Computed Tomography) or MRI (Magnetic Resonance Imaging). The observation device 700 is connected to the video control device 400 via a connection cable (not shown). Note that the observation device 700 is not limited to the device shown in FIG. 1.

The display device 900 includes an endoscope image display device 910 and an observation image device 920. The endoscope image display device 910 is a device capable of displaying images such as an LCD. The endoscope image display device 910 is connected to the video control device 400 via a display cable 911.

The observation image device 920 is a device capable of displaying an X-ray image. The observation image device 920 is connected to the video control device 400 via a display cable 921. When the observation device 700 includes a device for observing a patient by CT or MRI, the observation image device 920 includes a device capable of displaying CT images and MRI images.

Next, each device of the endoscope system 1000 will be described in detail.

### [Endoscope 100]

The endoscope 100 is a known side-viewing flexible endoscope, and includes a long insertion portion 110, an operation portion 120 provided at the proximal end of the insertion portion 110, and a universal code 150 extending from the operation portion 120. Note that the endoscope 100 may be a direct-view flexible endoscope.

The insertion portion 110 includes a distal rigid portion 111 provided at the distal end, a bendable bending portion 112 provided on the proximal side of the distal rigid portion 111, and a flexible tube portion 113 provided on the proximal side of the bending portion 112. The insertion portion 110 is formed with a treatment instrument channel 130 through which an endoscopic treatment instrument such as the stent delivery device 200 can be inserted.

A light guide 114, an imaging unit 115 having an imaging device such as a CCD, and a distal end opening 116 communicating with the treatment instrument channel 130 are provided on the side surface of the distal end rigid portion 111.

A raising base 117 is provided near the distal end opening 116 of the distal end rigid portion 111. A proximal end portion of the raising base 117 is rotatably supported by the distal end rigid portion 111. A raising base operation wire (not shown) fixed to the distal end of the raising base 117 extends through the insertion portion 110 to the operation portion 120.

The bending portion 112 can be bent vertically and horizontally. The distal end of the operation wire is fixed to the distal end side of the bending portion 112. The operation wire extends through the insertion portion 110 to the operation portion 120.

The operation portion 120 is provided with a knob 123 for operating the operation wire and the raising base operation wire, and a switch 124 for operating the imaging unit 115 and the like. The operator can bend the bending portion 112 in a desired direction by operating the knob 123.

The operation portion 120 is provided with a forceps opening (proximal end opening) 122 that communicates with the treatment instrument channel 130. The operator can insert an endoscopic instrument such as the stent delivery device 200 through the forceps port 122. A forceps plug 125 is attached to the forceps port 122 to prevent leakage of bodily fluids.

The universal cord 150 connects the endoscope 100 to the control device 500. An imaging signal imaged by the imaging unit 115 is transmitted to the video control device 400 via the universal code 150.

### [Stent Delivery Device 200]

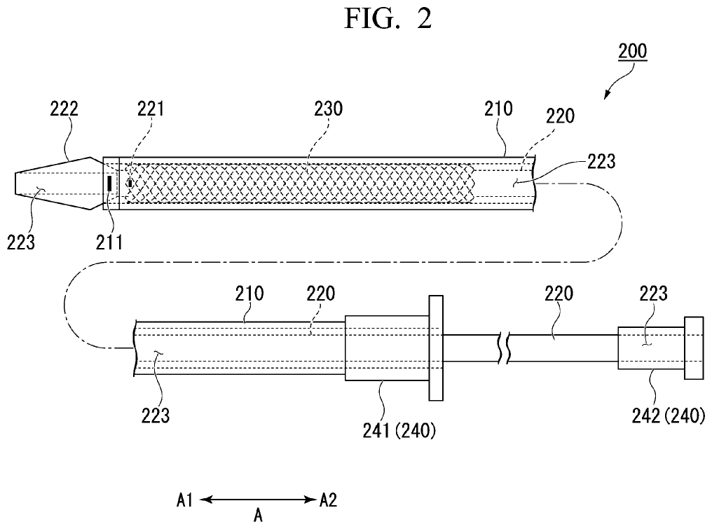

FIG. 2 shows a stent delivery device 200.

The stent delivery device 200 is elongated as a whole and includes an outer tubular member 210, an inner tubular member 220, a stent 230, and an operation portion 240.

In the following description, the side on which the stent delivery device 200 is inserted into the lumen of the patient P in the longitudinal direction A is referred to as a “distal end side (distal side) A1”, and the side of the operation portion 240 is referred to as a “proximal end side (proximal side) A2”.

The outer cylindrical member 210 is formed of resin or the like in a cylindrical shape and has flexibility. The outer cylinder member 210 can be inserted through the treatment instrument channel 130 of the endoscope 100. An outer tube marker 211 that is an X-ray opaque metal marker is provided at the distal end of the outer tube member 210.

The inner cylinder member 220 has an outer diameter smaller than the inner diameter of the outer cylinder member 210 and can be passed through the internal space (lumen) of the outer cylinder member 210. The inner cylindrical member 220 is made of resin or the like and has flexibility. A distal end 222 having an outer diameter larger than that of the outer cylindrical member 210 is provided at the distal end of the inner cylindrical member 220.

The stent 230 is a tubular self-expanding stent and is formed by weaving wires. The stent 230 is accommodated in the gap between the outer tubular member 210 and the inner tubular member 220 in a state in which the inner tubular member 220 is passed through the inner tubular member 220 and the diameter of the stent 230 is reduced. The stent 230 is locked by a locking portion 221 formed on the outer peripheral surface of the inner tubular member 220. As a result, the stent 230 is positioned relative to the inner cylinder member 220 in a reduced diameter state, and does not move in the longitudinal direction A relative to the inner cylinder member 220.

The wire forming the stent 230 is a superelastic alloy whose main material is NiTi. The superelastic alloy composed mainly of NiTi is not permanently deformed when it is woven, and the woven shape is memorized by applying a heat treatment in a woven state. The stent 230 may be a laser-cut type stent formed by cutting a metal tube with a laser.

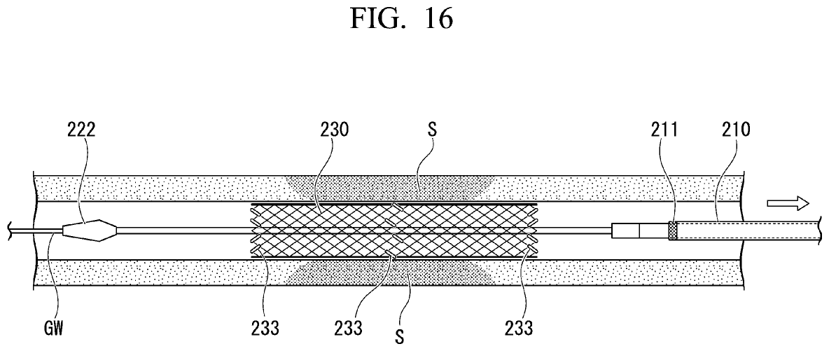

The stent 230 may have radiopaque markers 233 as shown in FIG. 16. The markers 233 shown in FIG. 16 are provided at the distal, central and proximal ends of stent 230.

The operation portion 240 is provided on the proximal end side A2 of the outer cylinder member 210 and the inner cylinder member 220, and is capable of moving the outer cylinder member 210 relative to the inner cylinder member 220 in the longitudinal direction A. The operation portion 240 has an outer cylinder operation portion 241 that drives the outer cylinder member 210 and an inner cylinder operation portion 242 that drives the inner cylinder member 220.

The operator can place the stent 230 by exposing the accommodated stent 230 by moving the outer tube operation portion 241 to the proximal side A2 with respect to the inner tube operation portion 242. The operator can also recapture the stent 230 by moving the outer tube operation portion 241 toward the distal end side A1 with respect to the inner tube operation portion 242.

A guide wire lumen 223 through which the guide wire GW is inserted is formed from the distal end 222 to the inner cylinder operation portion 242 of the operation portion 240 via the inner cylinder member 220.

### [Drive Device 300]

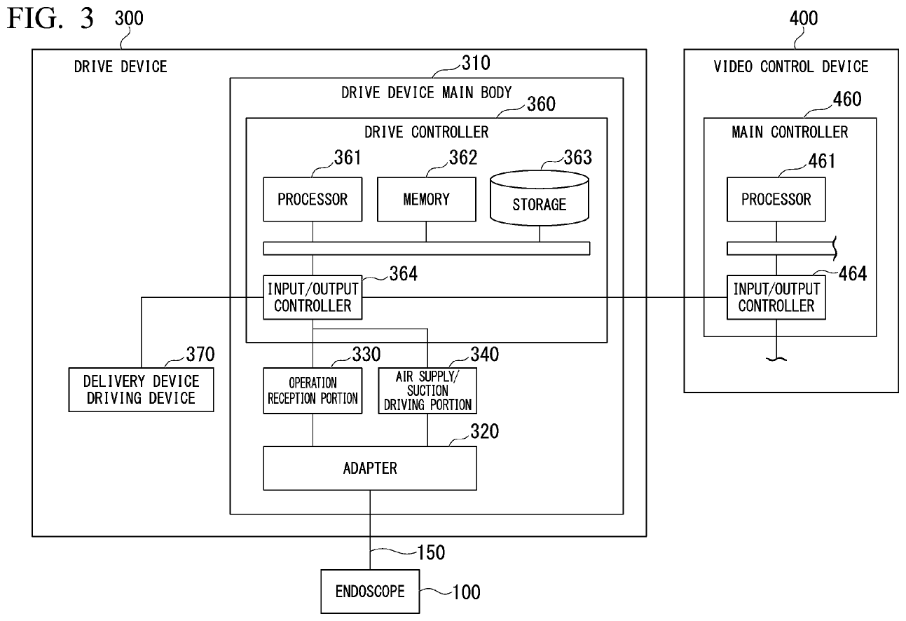

FIG. 3 is a functional block diagram of the drive device 300.

The drive device 300 includes a drive device main body 310 and a delivery device driving device 370. The drive device main body 310 and the delivery device driving device 370 may be an integrated device.

The drive device main body 310 has an adapter 320, an operation reception portion 330, an air supply/suction driving portion 340, and a drive controller 360.

The adapter 320 is an adapter to which the universal cord 150 of the endoscope 100 is detachably connected.

The operation reception portion 330 receives operation input from the operation portion 120 of the endoscope 100 via the universal code 150.

The air supply/suction driving portion 340 is connected to an air supply/suction tube through which the universal cord 150 is inserted. The air supply/suction driving portion 340 includes a pump or the like, and supplies air or liquid to the air supply/suction tube. Also, the air supply/suction driving portion 340 sucks air from the air supply/suction tube.

The drive controller 360 controls the drive device 300 as a whole. The drive controller 360 acquires the operation input received by the operation reception portion 330. The drive controller 360 controls the air supply/suction driving portion 340 and the delivery device driving device 370 based on the acquired operation input and the like.

The drive controller 360 includes a processor 361, a memory 362, a storage portion 363 capable of storing programs and data, and an input/output control portion 364. The drive controller 360 is a programmable computer. The functions of the drive controller 360 are implemented by the processor 361 executing programs. At least some functions of the drive controller 360 may be realized by dedicated logic circuits.

The input/output control portion 364 is connected to the operation reception portion 330, the air supply/suction driving portion 340, the delivery device driving device 370, the video control device 400, the input device (not shown), and the network device (not shown). Under the control of the processor 361, the input/output control portion 364 transmits and receives data and control signals to and from connected devices.

The drive controller 360 may further have components other than the processor 361, the memory 362, the storage portion 363, and the input/output control portion 364. For example, the drive controller 360 may further include an image calculation portion that performs part or all of the image processing and image recognition processing. By further having an image calculation portion, the drive controller 360 can execute specific image processing and image recognition processing at high speed. The image calculation portion may be mounted in a separate hardware device connected via a communication line.

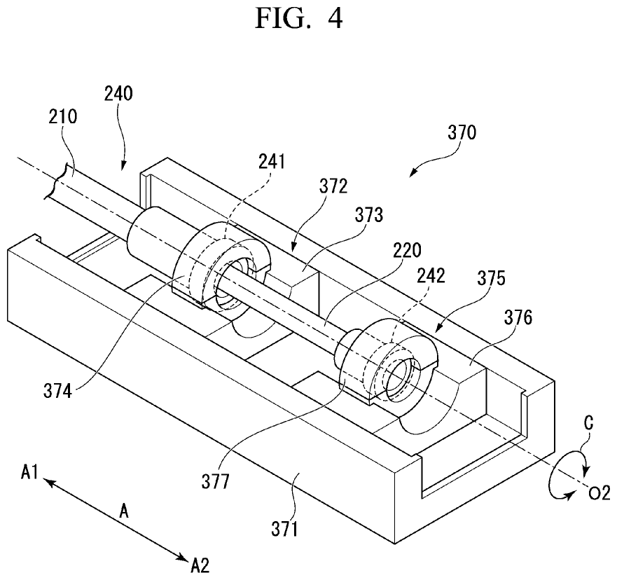

FIG. 4 is a diagram showing the delivery device driving device 370.

The delivery device driving device 370 is a device to which the operation portion 240 of the stent delivery device 200 is detachably connected. The delivery device driving device 370 can operate the connected operation portion 240 based on instructions from the drive controller 360.

The delivery device driving device 370 has a main body 371, an outer cylinder driving portion 372, and an inner cylinder driving portion 375.

The outer cylinder driving portion 372 is detachably fixed to the outer cylinder operation portion 241. The outer cylinder driving portion 372 has an outer cylinder forward/backward driving portion 373 and an outer cylinder rotating driving portion 374. The outer cylinder advancing/retreating driving portion 373 is driven by a motor or the like, and moves the outer cylinder operation portion 241 forward/backward in the longitudinal direction A with respect to the main body 371. The outer cylinder rotation driving portion 374 is driven by a motor or the like, and rotates the outer cylinder operation portion 241 about the central axis O2 in the longitudinal direction A with respect to the outer cylinder advancing/retreating driving portion 373.

The inner cylinder driving portion 375 is detachably fixed to the inner cylinder operation portion 242. The inner cylinder driving portion 375 has an inner cylinder advancing/retreating driving portion 376 and an inner cylinder rotating driving portion 377. The inner cylinder advancing/retreating driving portion 376 is driven by a motor or the like, and moves the inner cylinder operation portion 242 forward/backward in the longitudinal direction A with respect to the main body 371. The inner cylinder rotation driving portion 377 is driven by a motor or the like, and rotates the inner cylinder operation portion 242 about the central axis O2 in the longitudinal direction A with respect to the inner cylinder forward/backward driving portion 376.

### [Video Control Device 400]

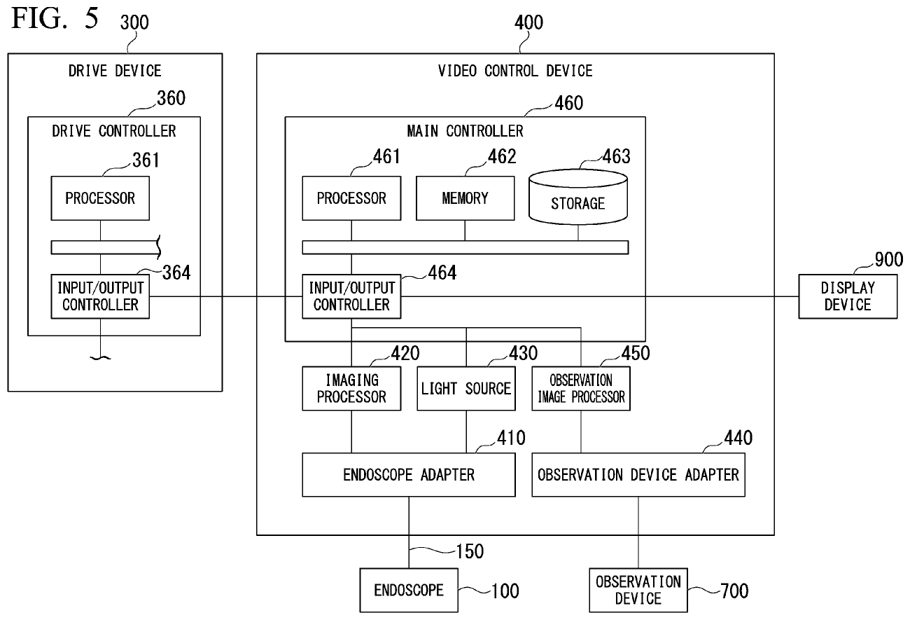

FIG. 5 is a functional block diagram of the video control device 400.

The video control device 400 includes an endoscope adapter 410, an imaging processing portion 420, a light source portion 430, an observation device adapter 440, an observation image processing portion 450, and a main controller 460.

The endoscope adapter 410 is an adapter to which the universal cord 150 of the endoscope 100 is detachably connected.

The imaging processing portion 420 acquires imaging signals from the imaging unit 115 of the endoscope 100 via the universal code 150. The imaging processing portion 420 converts the acquired imaging signal into a captured image.

The light source portion 430 is connected to a light cable through which the universal cord 150 is inserted. The light source portion 430 generates illumination light that irradiates the object to be imaged. The illumination light generated by the light source portion 430 is guided to the light guide 114 of the distal end rigid portion 111 of the endoscope 100 via a light cable or the like.

The observation device adapter 440 is an adapter to which a connection cable (not shown) connected to the observation device 700 is detachably connected.

The observation image processing portion 450 acquires the X-ray observation signal from the observation device 700 via the connection cable. The observation image processing portion 450 converts the acquired X-ray observation signal into an X-ray observation image. When the observation device 700 includes a device for observing a patient by CT, the observation image processing portion 450 converts the obtained CT observation signal into a CT image. When the observation device 700 includes a device for observing a patient by MRI, the observation image processing portion 450 converts the acquired MRI observation signal into an MRI image. Note that the observation device adapter 440 and the observation image processing portion 450 may be devices separated from the video control device 400.

The main controller 460 has a processor 461, a memory 462 into which a program can be read, a storage portion 463, and an input/output control portion 464. The main controller 460 is a computer capable of executing programs. The functions of the main controller 460 are implemented by the processor 461 executing programs. At least part of the functions of the main controller 460 may be realized by a dedicated logic circuit.

The storage portion 463 is a non-volatile recording medium that stores the above-described programs and necessary data. The storage portion 463 is composed of, for example, a ROM, a hard disk, or the like. A program stored in the storage portion 463 is read into the memory 462 and executed by the processor 461.

The input/output control portion 464 is connected to the imaging processing portion 420, the light source portion 430, the observation image processing portion 450, the drive device 300, the display device 900, the input device (not shown), and the network equipment (not shown). Under the control of the processor 461, the input/output control portion 464 transmits and receives data and control signals to and from connected devices.

The main controller 460 can perform image processing on the captured image acquired by the imaging processing portion 420 and the X-ray observation image acquired by the observation image processing portion 450. The main controller 460 can generate GUI images and CG images for the purpose of providing information to the operator S. The main controller 460 can display captured images, X-ray observation images, GUI images, and CG images on the display device 900.

The main controller 460 is not limited to an integrated hardware device. For example, the main controller 460 may be configured by separating part of it as a separate hardware device and then connecting the separated hardware device with a communication line. For example, the main controller 460 may be a cloud system that connects the separated storage portions 463 with a communication line.

The main controller 460 may further have components other than the processor 461, the memory 462, the storage portion 463 and the input/output control portion 464. For example, the main controller 460 may further have an image calculation portion that performs part or all of the image processing and image recognition processing. By further having an image calculation portion, the main controller 460 can execute specific image processing and image recognition processing at high speed. The image calculation portion may be mounted in a separate hardware device connected via a communication line.

### [Operation of Endoscope System 1000]

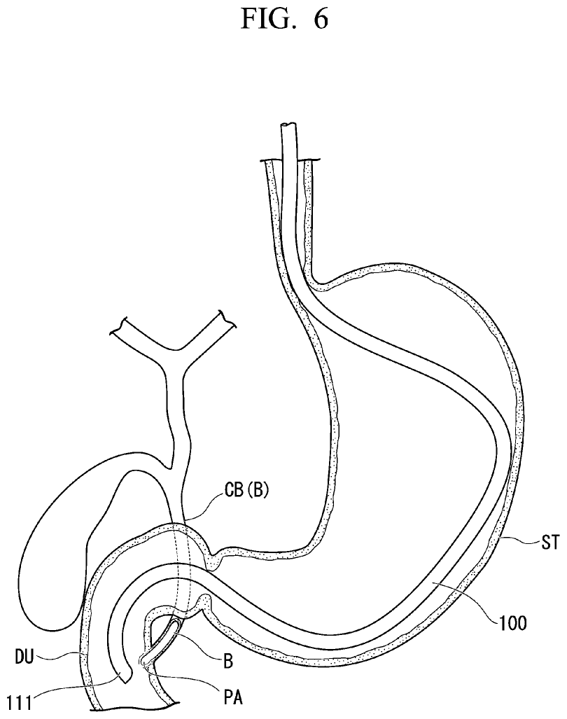

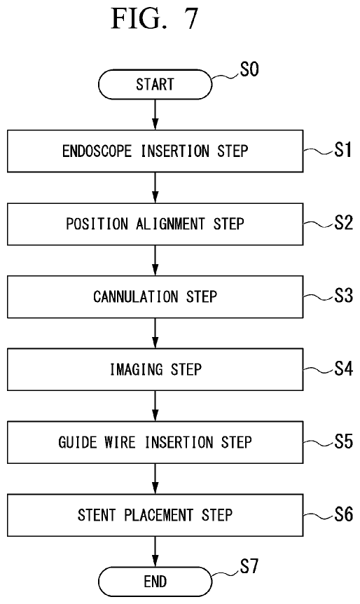

Next, the operation of the endoscope system 1000 according to this embodiment will be described. Specifically, a procedure for placing the stent 230 in the bile duct B by endoscopic retrograde cholangiopancreatography (ERCP) will be described. FIG. 6 shows a bile duct B in which a stent 230 is placed. FIG. 7 is a follow chart showing the steps of the procedure.

### <Step S1: Endoscope Insertion Step>

In step S1, the operator inserts the insertion portion 110 of the endoscope 100 into the patient's lumen through a natural opening such as the mouth. The operator bends the bending portion 112 by operating the knob 123 or the like as necessary. The operator inserts the distal rigid portion 111 of the endoscope 100 into the duodenum DU.

### <Step S2: Position Alignment Step>

In step S2, the operator adjusts the position of the distal rigid portion 111 of the endoscope 100 so that the papilla PA is within the imaging range of the imaging unit 115 of the endoscope 100.

### <Step S3: Cannulation Step>

The operator inserts a cannula from the papilla PA into the bile duct B in step S3. Specifically, a cannula is inserted into the treatment instrument channel 130 of the endoscope 100 to protrude from the distal end opening 116, and the cannula is inserted into the bile duct B.

### <Step S4: Imaging Step>

In step S4, the operator injects the contrast medium into the cannula to flow the contrast medium into the bile duct B through the cannula. The operator obtains an X-ray image showing the bile duct B and the like by performing X-ray imaging using the observation device 700. The operator acquires CT images and MRI images as necessary.

### <Step S5: Guide Wire Insertion Step>

The operator inserts the guide wire GW into the cannula, protrudes the guide wire GW from the cannula, and inserts the guide wire GW into the bile duct B in step S5. Next, the operator withdraws the cannula while leaving the guide wire GW in the bile duct B. Thereby, only the guide wire GW is left in the bile duct B.

### <Step S6: Stent Placement Step>

The operator inserts the stent delivery device 200 into the bile duct B along the guide wire GW in step S6. The control device 500 assists the stent placement step (step S6) as described below.

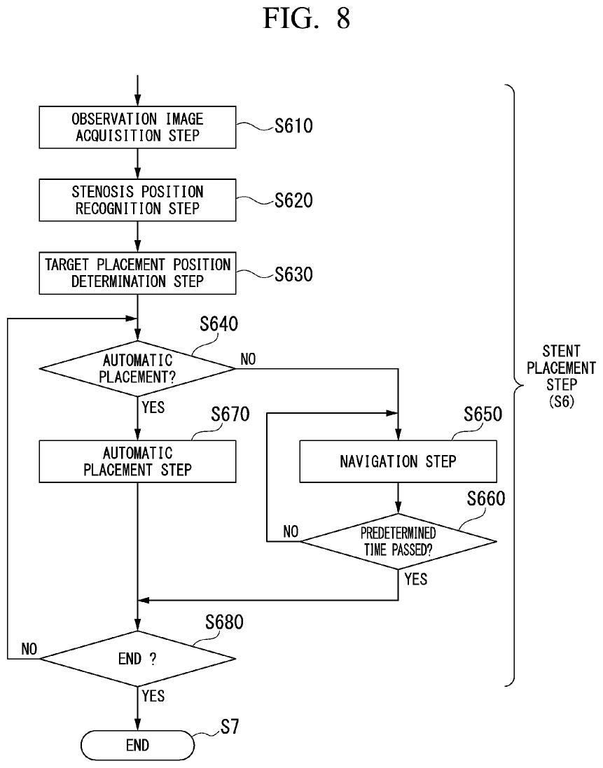

Hereinafter, description will be given along the control flowchart of the main controller 460 of the control device 500 in the stent placement step (step S6) shown in FIG. 8. When the operator inputs an operation start instruction to the control device 500, the main controller 460 performs initialization and then starts the following control. First, the main controller 460 (mainly processor 461) executes step S610.

### <Step S610: Observation Image Acquisition Step>

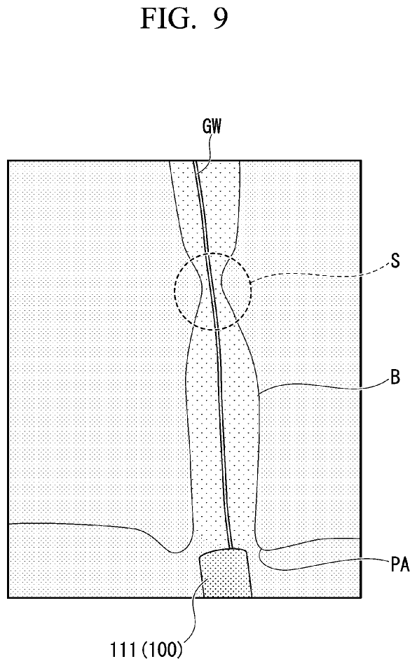

FIG. 9 is an X-ray image acquired by the observation image acquisition step.

In step S610, the main controller 460 acquires an X-ray image (observation image) showing the bile duct B and the like. The main controller 460 acquires CT images and MRI images as needed. The main controller 460 then executes step S620.

### <Step S620: Stenosis Position Recognition Step>

The main controller 460 recognizes the position of the stenosis S based on the observed image in step S620. Specifically, the main controller 460 recognizes a portion of the bile duct B in the observation image that has a smaller inner diameter than other portions as the stenosis S. The main controller 460 then executes step S630.

### <Step S630: Target Placement Position Determination Step>

In step S630, the main controller 460 determines a “target placement position TP” for placing the stent 230 in the stenosis S based on the observed image. Specifically, the main controller 460 determines the position of the stent 230 where the stenosis S is sandwiched between the distal end of the stent 230 and the proximal end of the stent 230 as the target placement position TP.

Generally, the main controller 460 determines the position of the stent 230 where the center of the stent 230 substantially coincides with the center of the stenosis S as the target placement position TP.

When the stenosis S is formed in the common bile duct CB, the main controller 460 may determine the position of the stent 230 at which the proximal end of the stent 230 protrudes from the papilla PA into the duodenum DU as the target placement position TP. By placing the stent 230 in a state in which the proximal end of the stent 230 protrudes from the papilla PA into the duodenum DU, the stent 230 can be prevented from entering the bile duct B after placement.

The main controller 460 may determine the target placement position TP of the stent 230 based on a model (database or learned model) in which the relationship between the observed image and the optimal target placement position TP of the stent 230 is learned by machine learning or the like.

After determining the target placement position TP of the stent 230, the main controller 460 executes step S640.

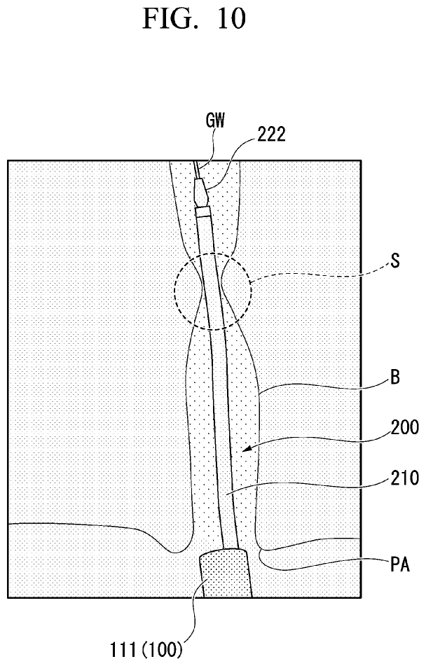

FIG. 10 is a diagram showing the stent delivery device 200 moved to the vicinity of the stenosis S.

The operator manipulates the stent delivery device 200 under X-ray fluoroscopy to move the stent delivery device 200 along the guide wire GW to the liver side and moves the portion containing the stent 230 in the stent delivery device 200 to the vicinity of the stenosis S.

### <Step S640: Automatic Placement Determination Step>

The main controller 460 determines whether to automatically place the stent 230 in step S640. Automatic placement means that the main controller 460 drives the delivery device driving device 370 to operate the operation portion 240 of the stent delivery device 200 to place the stent 230.

For example, the main controller 460 determines that the stent 230 is to be automatically placed when the operation portion 240 of the stent delivery device 200 is attached to the delivery device driving device 370. The main controller 460 may, for example, determine whether to automatically deploy the stent 230 based on an input from an input device such as a switch.

When automatically deploying the stent 230, the main controller 460 next executes step S670. If stent 230 is not to be auto-deployed, main controller 460 next executes step S650.

### <Step S650: Navigation Step>

The main controller 460 displays navigation information for navigating the operation of the stent delivery device 200 by the operator on the display device 900 in step S650. Specifically, the main controller 460 recognizes the position of the stent delivery device 200 from the observation image, and displays navigation information for navigating the operation of the stent delivery device 200 by the operator on the display device 900 together with the observation image.

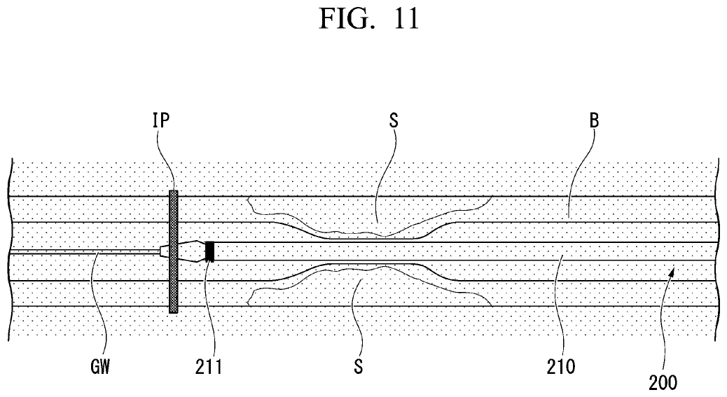

FIG. 11 is a display image of the display device 900 displaying navigation information.

The main controller 460, for example, calculates the “optimum position IP”, which is the optimum placement position of the stent delivery device 200 at which the stent 230 to be released is placed at the target placement position TP. The main controller 460 superimposes the optimum position IP on the display device 900 as navigation information. The optimum position IP shown in FIG. 11 is the position at which the outer tube marker 211 should be placed so that the released stent 230 is placed at the target placement position TP.

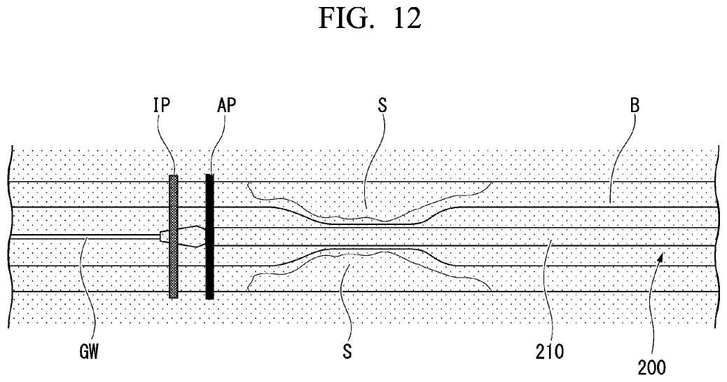

FIG. 12 is a display image of the display device 900 displaying other navigation information.

The main controller 460 may highlight the actual position AP of the outer cylinder marker 211 on the display device 900 as navigation information. The operator can easily grasp the positional deviation (gap) between the optimum position IP and the actual position AP of the outer cylinder marker 211. The main controller 460 may display the distance (numerical information) between the optimal position IP and the actual position AP of the outer cylinder marker 211 as navigation information.

When the stent 230 has the X-ray opaque marker 233, the main controller 460 may calculate the “optimum position IP”, which is the optimum release position of the stent 230 placed at the target placement position TP, and display the optimal position IP on the display device 900 as navigation information.

While watching the display image on which the optimal position IP is displayed, the operator moves the stent delivery device 200 so that the preset reference position (the outer tube marker 211 of the stent delivery device 200, the marker 233 of the stent 230, etc.) approaches the optimum position IP. After the reference position substantially coincides with the optimum position IP, the operator manually places the stent 230.

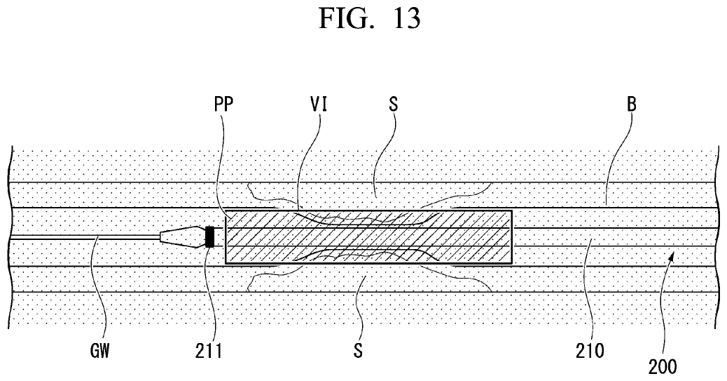

FIG. 13 is a display image of the display device 900 displaying other navigation information.

The main controller 460 calculates the “predicted placement position PP” to be placed when the stent 230 accommodated in the stent delivery device 200 is released, and superimpose a virtual image VI of the stent 230 placed at the predicted placement position PP on the display device 900 as navigation information.

While watching the display image showing the virtual image VI of the stent 230 placed at the predicted placement position PP, the operator moves stent delivery device 200 so that virtual image VI of stent 230 is optimally positioned at stenosis S. After that, the operator manually places the stent 230.

### <Step S660>

The main controller 460 repeatedly executes step S650 until a predetermined time elapses. The main controller 460 updates the navigation information based on the latest observation image (live data) acquired from the observation device 700 and displays it on the display device 900 in step S650 which is repeatedly performed. The main controller 460 executes step S680 after a predetermined period of time has elapsed.

### <Step S670: Automatic Placement Step>

In step S670, the main controller 460 drives the delivery device driving device 370 and operates the operation portion 240 of the stent delivery device 200 to automatically place the stent 230 at the target placement position TP.

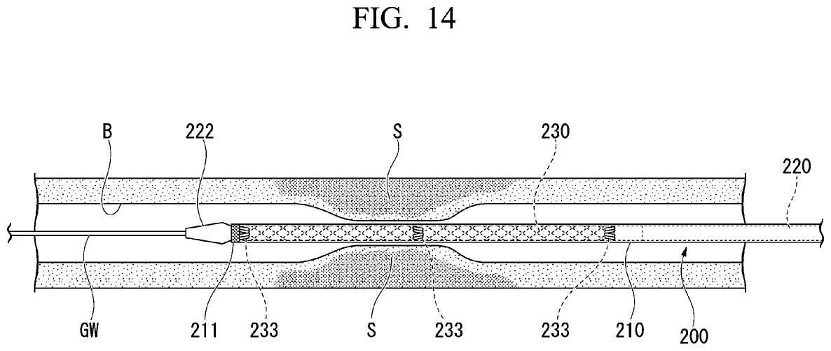

FIG. 14 is a diagram explaining the automatic placement step.

As in the navigation step, the main controller 460 calculates the “optimum position IP”, which is the optimum placement position of the stent delivery device 200 at which the stent 230 to be released is placed at the target placement position TP.

When the stent 230 has the X-ray opaque marker 233, the main controller 460 may calculate the “optimum position IP”, which is the optimum release position of the stent 230 placed at the target placement position TP, as in the navigation step.

The main controller 460 drives the delivery device driving device 370 by communicating with the drive controller 36) to operate the operation portion 240 of the stent delivery device 200. Specifically, the main controller 460 moves the stent delivery device 200 until a preset reference position (the outer cylinder marker 211 of the stent delivery device 200, the marker 233 of the stent 230, etc.) approximately matches the optimum position IP. The main controller 460 advances and retreats the entire stent delivery device 200 by driving the delivery device driving device 370 to simultaneously advance and retreat the outer cylinder driving portion 372 and the inner cylinder driving portion 375.

After the reference position substantially coincides with the optimum position IP, the main controller 460 drives the delivery device driving device 370 to release and place the stent 230. The delivery device driving device 370 exposes the accommodated stent 230 by moving the outer tube operation portion 241 to the proximal end side A2 with respect to the inner tube operation portion 242, and places the stent 230 therein.

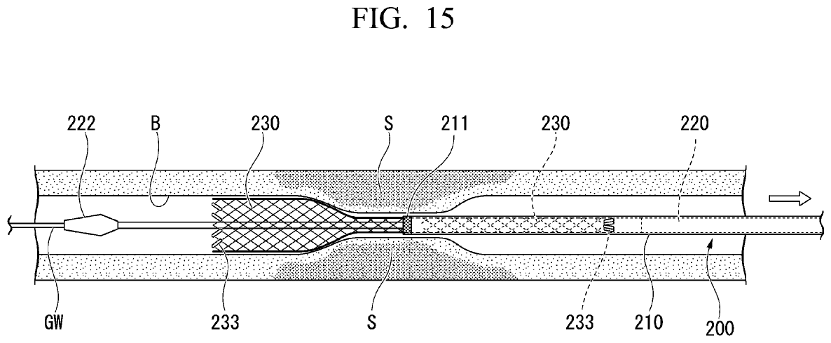

FIG. 15 is a diagram showing the stent 230 being deployed in the automatic deployment step.

The main controller 460 calculates the difference between the target placement position TP and the actually placed stent 230 based on the latest observation image (live data) acquired from the observation device 700. The main controller 460 can recognize the position of indwelling stent 230 by marker 233 provided on stent 230. The main controller 460 may grasp the position where the marker 233 is provided on the stent 230 based on the input product number of the stent 230. When the difference is large, the main controller 460 feedback-controls the delivery device driving device 370 so that the stent 230 is placed at the target placement position TP.

When the outer cylinder operation portion 241 is moved toward the proximal end side A2 with respect to the inner cylinder operation portion 242, in some cases, the inner cylinder member 220 advances toward the distal end side A1 because the friction between the outer cylinder member 210 and the treatment instrument channel 130 is large. In this case, the stent 230 is more likely to be indwelled on the distal end side A1 than the target indwelling position TP. Therefore, the main controller 460 retracts both the outer tubular member 210 and the inner tubular member 220 by the feedback control described above, and adjusts the position of the stent delivery device 200 so that the stent 230 is placed at the target placement position TP.

FIG. 16 is a diagram showing the stent 230 placed in the automatic placement step.

By performing the feedback control described above, the main controller 460 can reliably place the stent 230 at the target placement position TP. A higher frequency of feedback control can be applied.

The main controller 460 executes step S680 when the automatic placement of the stent 230 is completed or when an input to suspend the automatic placement of the stent 230 is received from the operator.

### <Step S680: End Determination Step>

In step S680, the main controller 460 determines whether the operator or the like has input to end the stent placement step (step S6). If the stent placement step (step S6) is not finished, the main controller 460 performs step S640 (automatic placement determination step) again.

The operator can repeatedly select whether to automatically place the stent 230 during the stent placement step (step S6). The operator manually placed the stent 230 based on the navigation without automatically placing the stent 230, but the procedure can be changed to automatically place the stent 230 midway through. Also, the operator has performed automatic placement of the stent 230, but this can be changed so as to perform manual placement of the stent 230 based on navigation midway through.

Note that if the patient's posture or the position of the observation device 700 changes, the main controller 460 restarts the process from step S610 and redetermines the “target placement position TP”.

When ending the stent placement step (step S6), the main controller 460 ends the control of the stent placement step (step S6). The operator withdraws the stent delivery device 200 excluding the stent 230 from the body.

A part or all of the control flowchart of the main controller 460 described above may be implemented by the drive controller 360.

According to the endoscope system (stent delivery system) 1000 of this embodiment, it is easy to place the stent at the target position.

As described above, the first embodiment of the present disclosure has been described in detail with reference to the drawings, but the specific configuration is not limited to this embodiment, and design changes etc. within the scope of the present disclosure are included. In addition, the constituent elements shown in the above-described first embodiment and modifications shown below can be combined as appropriate.

For example, the delivery device driving device 370 in the above embodiment may have a support portion formed of a highly rigid member so that the connected outer cylinder member 210 does not bend. The support portion of the delivery device driving device 370 supports the outer cylinder member 210 so that the path of the outer cylinder member 210 protruding from the forceps opening 122 is as straight as possible. By making the path of the outer cylinder member 210 linear, the path difference between the outer cylinder member 210 and the inner cylinder member 220 can be reduced. As a result, it is possible to prevent the inner cylinder member 220 from advancing toward the distal end side A1 when the outer cylinder operating part 241 is moved to the proximal end side A2 with respect to the inner cylinder operating part 242.

### ADDITIONAL NOTE

A stent delivery system, comprising:
a stent delivery device configured to carry a stent to a stenosis and indwell the stent;
an observation device configured to observe the stenosis;
one or more controllers, each comprising hardware, the one or more controllers being configured to:
   acquire an observation image from the observation device; and
      determine a target placement position for placing the stent in the stenosis based on the observation image, and
      control a driving device configured to drive the stent delivery device to place the stent at the determined target placement position.

The stent delivery system according to the above, wherein the one or more controllers being configured to calculate an optimal position, which is an optimal placement position of the stent delivery device at which the stent to be released is placed at the target placement position, and places the stent delivery device at the optimal position and releases the stent.

The stent delivery system according the above, wherein the one or more controllers being configured to calculate an optimal position, which is an optimum release position of the stent placed at the target placement position, and places the stent at the optimal position and releasing the stent.

The stent delivery system according to the above, wherein the one or more controllers being configured to disable placement of the stent by the driving device based on instructions from an operator.

The stent delivery system according to the above, wherein the one or more controllers being configured to recalculate the optimum position based on the observation image newly acquired from the observation device.

The stent delivery system according to the above, wherein the one or more controllers being configured to perform a feedback control on the driving device so that the stent is placed at the determined target placement position based on a difference between the determined target placement position and an actual position where the stent is placed.

The stent delivery system according to the above, wherein

the observation device is an X-ray fluoroscopy device, and

the observation image is an X-ray image.

The stent delivery system according to the above, wherein the one or more controllers being configured to determine a position of the stent where a distal end of the stent and a proximal end of the stent sandwich the stenosis as the target placement position.

The stent delivery system according to the above, wherein, when the determined target placement position is a lower bile duct, the one or more controllers being configured to determine a position of the stent sandwiching the stenosis between a distal end of the stent and a proximal end of the stent and a position at which the proximal end of the stent protrudes from the papilla as the target placement position.

A stent delivery method for placing a stent with a stent delivery device, the method comprising:
determining a target placement position for placing the stent in the stenosis based on an observation image acquired from an observation device that observes the stenosis; and
controlling a driving device using one or more controllers to drive the stent delivery device to place the stent at the target placement position.

The stent delivery method according to the above, wherein, the controlling comprises:
calculating an optimal position, which is an optimal placement position of the stent delivery device at which the stent to be released is placed at the determined target placement position, and
placing the stent delivery device at the optimal position to release the stent.

The stent delivery method according to the above, wherein the controlling comprises:
calculating an optimum position, which is the optimum release position of the stent placed at the determined target placement position;
placing the stent at the optimum position; and
releasing the stent.

The stent delivery method according to the above, wherein, further comprising invalidating placement of the stent by the driving device based on instructions from an operator.

The stent delivery method according to the above, wherein, the controlling comprises feedback-controlling the driving device so that the stent is placed at the determined target placement position based on a difference between the determined target placement position and an actual position where the stent is placed.

What is claimed is:

1. A stent delivery system, comprising:
a stent delivery device configured to carry a stent to a stenosis and indwell the stent;
an observation device configured to observe the stenosis; and
one or more processors, each comprising hardware, the one or more processors being configured to:
acquire an observation image from the observation device; and
determine a target placement position for placing the stent in the stenosis based on the observation image.

2. The stent delivery system according to claim 1, further comprising:
a display,
wherein the one or more processors is configured to control the display to display each of navigation information regarding the determined target placement position and the observation image.

3. The stent delivery system according to claim 2, wherein the one or more processors is configured to:
calculate an optimal position, which is an optimal placement position of the stent delivery device at which the stent to be released is placed at the target placement position, and
control the display to display the optimal position as the navigation information.

4. The stent delivery system according to claim 2, wherein the one or more processors is configured to:
calculate an optimal position, which is an optimum release position of the stent placed at the target placement position, and
control the display to display the optimal position as the navigation information.

5. The stent delivery system according to claim 2, wherein the one or more processors is configured to:
calculate a predicted placement position when the stent housed in the stent delivery device is released, and
control to the display to display the predicted placement position as the navigation information.

6. The stent delivery system according to claim 5, wherein the one or more processors is configured to control the display to display a virtual image of the stent superimposed at the predicted placement position.

7. The stent delivery system according to claim 3, wherein the one or more processors is configured to recalculate the optimum position based on the observation image newly acquired from the observation device.

8. The stent delivery system according to claim 5, wherein the one or more processors is configured to recalculate the predicted placement position based on the observation image newly acquired from the observation device.

9. The stent delivery system according to claim 1, wherein
the observation device is an X-ray fluoroscopy device, and
the observation image is an X-ray image.

10. The stent delivery system according to claim 1, wherein the one or more processors is configured to determine a position of the stent where a distal end of the stent and a proximal end of the stent sandwich the stenosis as the target placement position.

11. The stent delivery system according to claim 1, wherein, when the determined target placement position is the lower bile duct, the one or more processors is configured to determine a position of the stent sandwiching the stenosis between a distal end of the stent and a proximal end of the stent and where a proximal end of the stent protrudes from a papilla, as the target placement position.

12. A stent delivery method for placing a stent with a stent delivery device, the method comprising:
acquiring an observation image from an observation device that observes the stenosis;
determining a target placement position for placing the stent in a stenosis based on the observation image.

13. The stent delivery method according to claim 12, further comprising:
providing navigation information about the determined target placement position together with the observation image.

14. The stent delivery method according to claim 13, wherein providing the navigation information comprises:
calculating an optimal position, which is an optimal placement position of the stent delivery device at which the stent to be released is placed at the determined target placement position; and
providing the optimal position as the navigation information.

15. The stent delivery method according to claim 13, wherein providing the navigation information comprises:
calculating an optimum position, which is an optimum release position of the stent placed at the determined target placement position, and
providing the optimum position as the navigation information.

16. The stent delivery method according to claim 13, wherein providing the navigation information comprises:
calculating a predicted placement position to be placed when the stent housed in the stent delivery device is released; and
providing the predicted placement position as the navigation information.

17. The stent delivery method according to claim 16, wherein providing the navigation information comprises superimposing a virtual image of the stent placed at the predicted placement position on the observation image.

18. A control device comprising:
one or more processors comprising hardware, the one or more processors being configured to:
determine a target placement position for placing a stent in a stenosis based on an observation image acquired from an observation device that observes the stenosis.

19. The control device according to claim 18, wherein the one or more processors is configured to determine a position of the stent where a distal end of the stent and a proximal end of the stent sandwich the stenosis as the target placement position.

20. The control device according to claim 18, wherein, when the determined target placement position is the lower bile duct, the one or more processors is configured to determine a position of the stent sandwiching the stenosis between a distal end of the stent and a proximal end of the stent and where a proximal end of the stent protrudes from a papilla, as the determined target placement position.
